(19) United States
(12) Patent Application Publication
(10) Pub. No.: US 2005/0208671 A1
(43) Pub. Date: Sep. 22, 2005

(54) Method of analyzing the ratio of activation of terminals of polyoxyalkylene derivatives

(75) Inventors: Nobuko Takano, Yokohama-shi (JP); Kouzoh Sanchika, Kawasaki-shi (JP)

Correspondence Address: OLIFF & BERRIDGE, PLC, P.O. BOX 19928, ALEXANDRIA, VA, 22320, US

(73) Assignee: NOF CORPORATION, Shibuya-ku (JP)

(21) Appl. No.: 11/019,189

(22) Filed: Dec. 23, 2004

(30) Foreign Application Priority Data
Dec. 26, 2003 (JP) 2003-433256

(51) Int. Cl. G01N 33/00

(52) U.S. Cl. 436/86

(57) ABSTRACT

An object the invention is to provide a method of analyzing the activation ratio of terminals of a polyoxyalkylene derivative so that the ratio can be accurately measured at a high precision even when the polyoxyalkylene derivative has a high molecular weight. The activation ratio of terminal of a polyoxyalkylene derivative having a terminal active group capable of bonding with a biologically active substance having a molecular weight of 1000 to 100000 is analyzed. The active group is labeled using a labeling reagent having an ionic functional group. The polyoxyalkylene derivative is then analyzed by means of liquid chromatography using an ion-exchange column and an RI detector outputting a chromatogram. The activation ratio of terminal is obtained based on percentage of an area of a peak corresponding to the active group in the chromatogram.

This application claims the benefit of Japanese Patent Application 2003-433256, filed on Dec. 26, 2003, the entirety of which is incorporated by reference.

### BACKGROUND OF THE INVENTION

1. Field of the Invention

The present invention relates to a method of analyzing the ratio of activation of terminal of polyoxyalkylene derivatives. More specifically, the present invention relates to a method of measuring the activation ratio of a terminal of polyoxyalkylene derivative having an active group at the terminal, for applying the derivative as polyoxyalkylene modifiers for polypeptides, biologically active proteins, enzymes or the like and polyoxyalkylene modifier for drug delivery systems (DDS) such as biodegradable hydrogels, liposomes, polymer micelles or the like.

2. Related Art Statement

Recently, it has been developed a polymer compound for use in medical applications including a polyoxyalkylene derivative whose terminal is activated. The main applications include modifiers for polypeptides, biologically active proteins, enzymes or the like and drug delivery systems (DDS) such as biodegradable hydrogels, liposomes, polymer micelles or the like.

In the application of the modifiers, a biologically active substance is chemically modified with a polyoxyalkylene derivative, which is an amphipathic polymer, to increase the molecular weight and to improve the solubility. It is thus possible to reduce the immunogenicity, antigenicity and toxicity, to improve the stability of the drug and to lengthen the residence time in the body.

The polyoxyalkylene derivative has an active groups, at the terminal of polyoxyalkylene chain, capable of chemically bonding functional groups present on the surface of protein or the like to be modified, such as amino, mercapto or carboxyl group or unsaturated bond. For example, the polyoxyalkylene has an active group, at the terminal of the polyoxyalkylene chain, such as aldehyde, acetal, p-nitrophenyl or N-hydroxy succinimide group for modifying amino group, mercapto, maleimide, allyl or N-hydroxy succinimide group for modifying mercapto group, mercapto or amino group for modifying carboxyl group and mercapto group for modifying unsaturated bond.

Particularly when a low molecular weight drug or peptide is modified, however, the number of the reactive functional groups to be bonded with the polyoxyalkylene derivative is small so that the effect of improving the solubility is not feasible. Further, if a drug or peptide is modified with a number of polyoxyalkylene derivatives, the active sites of the drug or peptide is occupied with them, so that the characteristic functions or drug action cannot be sufficiently obtained. Recently, it has been used a polyoxyalkylene derivative having a higher molecular weight for effectively obtaining the effects with a minimum number of modified sites for avoiding the deterioration of the characteristic function or drug action of a biologically active substance.

On the other hand, a biodegradable hydrogel is used as a controlled release delivery system of drugs. That is, a biologically active and insoluble substance is incorporated into the biodegradable hydrogel formed using a biocompatible polymer such as a polyoxyalkylene derivative to utilize the biodegradability to control the release of the active substance. It is thus possible to control the level of the active component in the blood to provide more excellent effect, safety and convenience for the patient.

The polyoxyalkylene derivatives used in the biodegradable hydrogel usually has branches each having a terminal, which is cross-linked with another polyoxyalkylene derivative or the terminal functional group of the biocompatible polymer to form a network-like structure. The cross linked bondings are decomposed to release the biologically active substance. The rate of the decomposition depends on the number of terminal active groups of the polyoxyalkylene derivative, so that the release of the biologically active substance can be controlled.

The polyoxyalkylene derivative having such terminal active groups is synthesized by binding active groups with the terminal hydroxyl group of a polyoxyalkylene (activation of terminals). The reaction ratio in the terminal activation reaction is called “activation ratio of terminal”. In other words, “activation ratio of terminal” is a percentage of the number of active groups bonded with the terminal hydroxyl groups with respect to the number of terminal hydroxyl groups of a polyoxyalkylene derivative.

In the polyoxyalkylene derivative in use for a modifier, it is required a high reactivity with a drug. The activation ratio of terminal is required to be very high. It is thus required an analyzing method of evaluating the polyoxyalkylene derivative accurately and at a high precision.

In the application of the biodegradable hydorgel or the like, the rate of the degradation can be controlled with the number of the terminal active groups of the polyoxyalkylene derivative. It is required to accurately measure the activation ratio of terminal for accurately control the release of biologically active substances.

Further, the biological activity and safety of medicaments depend on impurities contained in the drug formulations. It is thus necessary to reduce the contents of impurities each having a molecular weight or a number of active groups different from those of a target compound. It is further necessary to specifically quantify only the target polyoxyalkylene derivative separately from the impurities.

As described above, in designing a drug formulation using a polyoxyalkylene derivative having an activated terminal, the activation ratio of terminal is one of the most important item for analysis. It is thus important that the ratio can be measured accurately and at a high precision with specificity for avoiding the influence of the impurities.

A titration method has been known for a long time for measuring the activation ratio of terminal of a polyoxyalkylene derivative. The method is, however, problematic in that the analytical error is generally large and the influence of the error is larger as the molecular weight of a sample for analysis is larger.

Recently, ¹H-NMR method is popular as a method of analyzing most of the activation ratio of terminal of polyoxyalkylene derivative because the analysis can be easily performed in a short time. For example, the ratio of activation of terminals of polyoxyalkylene derivative, whose terminals are replaced with maleimide groups, was calculated based on the measured integrated value and to the theoretical integrated value of hydrogen peak of the maleimide group (United states Patent publication No. 2001-44526A).

Another method frequently used includes a method of labeling terminal active groups to be analyzed with a colorimetric reagent, measuring the absorbance at a specific wavelength and calculating the activation ratio using the absorbance and a calibration curve prepared using a standard sample in advance (absorbance spectroscopy method). For example, as a method of determining terminal mercapto groups, it is well known a method of reacting the mercapto groups with a calorimetric reagent such as 2,2-dithiopydirine or 4,4-dithiopyridine and measuring the absorbance at a wavelength of 410 nm. The method was applied for a polyoxyalkylene derivatives (Shmuel Zalipsky, Int. J. Peptide Protein Res. 30, 198 7, 740). Polyoxyalkylene derivative having terminal p-nitrophenyl carbamate groups is fully hydrolyzed in a basic solution to release p-nitrophenol, which is then quantified by measurement of absorbance at a wavelength of 400 nm so that the activation ratio of terminal is calculated (F. M. Veronese, et al, Applied Biochemistry and Biotechnology, 11, 141 (1985)). Similarly, in the case of polyoxyalkylene derivative having terminal aldehyde group, it is applied absorbance spectroscopy method using Schiff reagent (J. Milton Harris, et al, Polymer Chemistry Edition, 22, 341(1984)).

### SUMMARY OF THE INVENTION

When the activation ratio of terminal of a polyoxyalkylene derivative is measured by ¹H-NMR method, the intensity of multiplet peaks corresponding with protons in the polyoxyalkylene chain is larger as the molecular weight of the analyzed sample is larger, resulting in influences such as an increase of noise and on the shift of the base line. The error in measuring the activation ratio of terminal becomes considerably larger as the molecular weight of the polyoxyalkylene derivative is larger.

On the other hand, according to the absorbance spectroscopy method, the analytical error is generally lower compared with ¹H-NMR method and is not increased when the molecular weight of the polyoxyalkylene derivative is large. The activation ratio in the method is, however, calculated based on molar absorbance coefficient and molecular weight of the analyzed sample. Since the molecular weight of the sample is determined based on hydroxyl value or GPC analysis in advance, the error in the molecular weight results in a considerable analytical error in the activation ratio. Further, when an impurity having activated terminal and having a molecular weight lower or higher than the target molecular weight is present, the measured activation ratio may be considerably deviated from the true activation ratio.

Usually when a polyoxyalkylene derivative is synthesized, it is used a method of bonding a lower molecular weight compound having an active group with terminal hydroxyl groups of polyoxyalkylene as a raw material. The reaction product often contains residue of the lower molecular weight compound having an active group. When the activation ratio of terminal of this kind of sample for analysis is measured by means of the absorbance spectroscopy method, however, the colorimetric reagent also reacts with the residual low molecular weight compound having the active groups. The thus obtained activation ratio is made higher than the true ratio.

Further, the activation ratio of terminal measured by ¹H-NMR or absorbance spectroscopy method is shown only as the ratio of the activated functional groups with respect to the total number of terminal functional groups. For example, in the case of a polyoxyalkylene derivative having two or more active groups equivalent and structurally symmetrical with each other, the ratio of each number of the activated groups cannot be obtained. Moreover, when the sample contains an impurity having the same active group and the different molecular weight as the target compound, such impurity cannot be distinguished in the measurement. For example, when a polyoxyalkylene derivative having an active group at one terminal is to be produced, a byproduct may be generated, in many cases, having a molecular weight larger by two fold than that of the target compound and having active groups at the one and the other terminals. Such byproduct having active groups at both terminals may induce cross-linking reaction in the modification reaction with a biologically active compound and thus to be avoided. It is, however, not possible to determine the ratio separately from that of the target compound according to the above reasons.

An object of the present invention is to provide a method of analyzing the activation ratio of terminal of a polyoxyalkylene derivative so that the ratio can be measured at a high precision even when the polyoxyalkylene derivative has a high molecular weight or when an activated substance different from the target compound is present.

The present invention provides a method of measuring an activation ratio of terminal of a polyoxyalkylene derivative having a terminal active group capable of chemically bonding with a biologically active substance and having a molecular weight of 1000 to 100000, said method comprising the steps of:

labeling the terminal active group using a labeling reagent having an ionic functional group;

then analyzing said polyoxyalkylene derivative by means of liquid chromatography using an ionic exchange column and an RI detector outputting a chromatogram; and

obtaining an activation ratio of terminal based on a percentage of an area of a peak corresponding with the terminal active group in the chromatogram.

The present invention is an epoch-making analytical method for providing the following advantageous effects in a measurement of the activation ratio of terminal of a polyoxyalkylene derivative. The invention has a possibility of establishing a standard analytical procedure in an area of medications or the like and considerably useful in the industry.

(1) It is possible to measure the ratio accurately and at a high precision in the case of a polyoxyalkylene derivative having a high molecular weight.

(2) It is possible to prevent the influence on the determination by an impurity of a low molecular weight, when such impurity is present in a sample of a polyoxyalkylene derivative.

(3) When an impurity having terminal active groups and a molecular weight different from that of a target molecular weight is contained in a sample of a polyoxyalkylene derivative, the derivative having the target molecular weight and impurity can be separately and independently measured.

(4) When a polyoxyalkylene derivative has a plurality of terminal active groups (a plurality of activation ratios of terminals), the ratios of the activated functional groups can be independently measured.

### BRIEF DESCRIPTION OF THE DRAWINGS

FIG. 1 shows the results of RI chromatogram obtained by measurement of liquid chromatography according to example 1.

FIG. 2 shows the results of UV chromatogram obtained by measurement of liquid chromatography according to example 1.

FIG. 3 shows the results of RI chromatogram obtained by measurement of liquid chromatography of an experimental sample (7-1) according to example 3.

FIG. 4 shows the results of RI chromatogram obtained by measurement of liquid chromatography of an experimental sample (7-2) according to example 3.

FIG. 5 shows the results of GPC measurement according to example 5.

FIG. 6 shows the results of RI chromatogram obtained by measurement of liquid chromatography according to example 5.

FIG. 7 shows the results of RI chromatogram obtained by measurement of liquid chromatography according to example 6.

### PREFERRED EMBODIMENTS FOR CARRYING OUT THE INVENTION

The present invention will be described below in detail.

The polyoxyalkylene bone structure of a polyoxyalkylene derivative subjected to the analysis according to the present invention may be either of straight chain or branched chain. According to the analytical method of the present invention, particularly in the case of a polyoxyalkylene derivative having a plurality of terminal active groups, the ratios of the polyoxyalkylene derivatives having different numbers of terminal active groups can be independently measured, unlike prior ¹H-NMR or absorbance spectroscopy method. It is thus preferred that the polyoxyalkylene derivative has two or more terminal active groups. Although the upper limit of the number of the terminal active groups is not particularly defined, for example, the ratios may be analyzed even when the derivative has up to 14 terminal active groups. Typically, the number of terminal active groups may be 2, 3 or 4.

The polyoxyalkylene bone structure may preferably be composed of an oxyalkylene group having 2 to 4 carbon atoms, and includes oxyethylene, oxypropylene, oxybutyrene and oxytetramethylene groups, which may be used alone or in combination. When two or more kinds of oxyalkylene groups are added, they may be bonded in blocks or ramdomly. Further, for performing the measurement of the analytical method at a high precision, the polyoxyalkylene derivative to be analyzed may preferably be water soluble. For this, oxyethylene group may preferably occupy 50 mole percent or more, more preferably 80 mole percent or more, and most preferably 100 mole percent of the oxyalkylene bone structure of the polyoxyalkylene derivative.

The molecular weight of the polyoxyalkylene derivative is 1000 to 100000 and preferably 5000 to 100000. According to the present invention, the activation ratio of the terminal of the polyoxyalkylene derivative having a high molecular weight can be analyzed at a high precision, unlike prior arts of titration and ¹H-NMR methods. For example, in the case of a straight chain polyoxyalkylene derivative having an active group at one terminal, the analytical error becomes considerable in the titration method when the molecular weight is 1000 or more. The advantageous effects of the present invention is thus important.

The polyoxyalkylene derivative to be analyzed according to the present invention have an active group, at the terminal, which specifically reacts with a biologically active substance such as a protein, a polypeptide or drug. The terminal active group includes aldehyde, nitrophenyl carbamate, mercapto, maleimide, allyl, amino, carboxyl groups etc., and may preferably be aldehyde, nitrophenyl carbamate, mercapto, maleimide or allyl group. These active group generally does not require the addition of a reaction catalyst and easily reacts with a biologically active substance to be modified upon mixing to provide a stable reaction product. Further, in the case of the polyoxyalkylene derivative having two or more terminal active groups, they may have different terminal active groups.

According to the method of analysis according to the present invention, a labeling reagent having an ionic functional group is used to label the terminal active group of the polyoxyalkylene derivative.

The ionic functional group includes carboxyl and amino groups. The corresponding labeling reagent have chemical structures shown in formulae 5 and 6, respectively. Each of the structures shown in the formulae (5) and (6) may have two or more ionic functional groups.
X—Y—COOH (5)
X—Y—NH₂ (6)

The “X” group may be any functional group as far as the functional group is capable of specifically reacting with the terminal active group of the polyoxyalkylene derivative. Specifically, when the terminal active group is maleimide or allyl group, “X” group of the labeling reagent may preferably be mercapto group. When the terminal active group is mercapto group, the “X” group of the labeling reagent may preferably be maleimide or ally group. When the terminal active group specifically reacts with amino group, for example aldehyde or p-nitrophenyl carbamate group, the labeling reagent of formula (6) may induce cross reaction and thus not preferable. The labeling reagent of formula (5) where the “X” group is amino group is preferably used in this case.

Any of saturated and unsaturated hydrocarbons having 1 to 24 carbon atoms may be used at the connecting point (Y) of the “X” group and carboxyl or amino group. Saturated or unsaturated hydrocarbon having 1 to 12 carbon atoms is preferably used, and more preferably unsaturated hydrocarbon having ultraviolet or fluorescent absorbance is more preferably used. When the number of carbon atoms is larger than 12, the labeling reaction may not be easily proceeded. When the “Y” group is an unsaturated hydrocarbon group having ultraviolet absorbance or fluorescent absorbance, ultraviolet detector (UV) or a fluorescent detector may be simultaneously used in HPLC analysis after the labeling reaction. It is thus possible to perform the analysis at a still higher sensitivity. The unsaturated hydrocarbon having ultraviolet or fluorescent absorbance includes phenyl, naphthyl, antracenyl, acridinyl groups etc..

The labeling reagent (5) having carboxyl group specifically includes the followings. In the case of terminal aldehyde or acetal group, the reagent includes amino acid such as glycine, alanine, phenylalanine, tyrosine or the like and p-aminobenzoic acid, and may preferably be glycine or p-aminobenzoic acid. In the case of terminal nitophenyl carbamate or N-hydroxy succin imide group, the reagent includes glycine, alanine, phenylalanine, tyrosine, aminocapronic acid and p-aminobenzoic acid, and may preferably be glycine, alanine or aminocapronic acid. In the case of terminal mercapto group, the reagent includes maleimide propionic acid. In the case of terminal maleimide or allyl group, the reagent includes mercaptopropionic acid, mercaptoacetic acid, mercaptonaphthyl acetic acid, thiosalicylic acid etc..

The labeling reagent (6) having amino group specifically includes the followings. In the case of terminal mercapto group, the reagent includes anilinonaphthyl maleimide, allylamine, 2-methyl allyl amine or the like. In the case of terminal maleimide or allyl group, thioethanol amine hydrochloride etc. are listed.

In the labeling reaction applied in the present invention, it is important that the reaction proceeds quantitatively to produce s stable labeled compound without the decomposition and side reaction, for accurately measuring the activation ratio of terminal. It is further preferred that the operation is easy to perform. The conditions for the labeling reaction will be described further in detail.

The amount of the added labeling reagent is not limited as far as it is excessive with respect to the amount of the polyoxyalkylene derivative for performing a quantitative labeling reaction. The amount of the labeling reagent may preferably be 5 to 50 equivalent and more preferably 5 to 20 equivalent with respect to that of the polyoxyalkylene derivative. Further, in the case of the polyoxyalkylene derivative having difunctional or multi functional terminal active groups, the excessive amount may preferably be adjusted in response to the number of the functional groups in the molecule.

Optionally, a basic catalyst such as triethyl amine or pyridine may be also added. Particularly in the case of terminal aldehyde group, a reducing reagent such as sodium borohydride or sodium cyanoborohydride may be added with the labeling reagent in an amount of 1 equivalent or larger of that of the labeling reagent to perform reducing alkylation reaction. It is thus possible to obtain an extremely stable labeled compound.

The reaction solvent includes water, a buffer solution, an organic solvent or the combination thereof, depending on the solubility and reactivity of the labeling reagent.

The kind and pH of the buffer solution is optional and may be decided on the reactivity of the polyoxyalkylene derivative and labeling reagent. For example, when reducing alkylation reaction is used for the derivative having terminal aldehyde group, it is preferred to perform the reaction at a pH lower than pKa (dissolution constant) of amino acid of the labeling reagent used.

The concentration of salt of the buffer solution is not limited and may preferably and normally be 10 to 500 mM and more preferably be 50 to 300 mM. The buffering action may not be obtained at the concentration of salt lower than 10 mM when excessive amount of the labeling reagent is used.

The organic solvent is not limited as far as the polyoxyalkylene derivative and labeling reagent are soluble in the solvent. The organic solvent includes methanol, ethanol, acetonitrile, 2-methoxy ethanol, dioxane, methylene chloride, chloroform, benzene, toluene etc.. When the active group is subjected to hydrolysis in a short time in water or buffer solution, for example terminal N-hydroxy succinimide group or terminal p-nitrophenyl carbamate group, only an organic solvent may be used.

The amount of the solvent used for the reaction is not limited, and may normally and preferably be 10 to 1000 weight percent, and more preferably be 20 to 500 weight percent with respect to the weight of the polyoxyalkylene derivative. When the amount is lower than 10 weight percent, the viscosity of the solution is increased to reduce the reactivity. Further in this case, the terminal aldehyde groups may perform condensation reaction or the terminal mercapto groups may generate disulfide bonds between the terminal mercapto groups. Such reactions may be a cause of preventing the labeling reaction. When the amount of the solvent exceeds 1000 weight percent, the reactivity may be reduced.

The temperature and time period for the reaction is optional and such conditions may be decided depending on the labeling reaction.

When the terminal maleimide group is to be detected, and when the labeling reagent having maleimide group is used for detecting the terminal mercapto group, the labeling reaction is performed in shading condition for preventing polymerization with light.

Although the reaction solution containing the polyoxyalkylene derivative labeled according to the above procedure may be used as a sample subjected to high performance liquid chromatography to perform rapid and easy analysis, the reaction solution may preferably be subjected to desalting process using a gel filtration chromatography. The kind of the gel filtration chromatography is not limited as far as it is capable of separating a low molecular weight substance having a molecular weight of 1000 or lower. The desalting process is performed according to the following procedure. The gel filtration column is equilibrated with buffer solution for use as an eluent for the subsequent high performance liquid chromatography, and the reaction solution after the labeling is then added. An eluent is then added to take a fraction containing high molecular weight substances eluted first, which is taken as an analytical sample. It is possible to remove the reagents of low molecular weights such as the excessive labeling reagent left in the reaction solution and to prevent the adsorption and contamination of the ionic exchange column by performing the desalting procedure. Further in the analysis of using a high performance liquid chromatography, a ghost peak may generally be eluted interfering the quantification when the eluent and the dissolving solvent of the sample are different with each other. The reaction solvent of the sample is exchanged with the eluent by performing the desalting, so that the ghost peak can be prevented to perform accurate quantification.

The polyoxyalkylene derivative with ionic property given by the labeling is then separated with a high performance liquid chromatography using an ion-exchange column to perform the measurement. The conditions for the measurement will be described below in detail.

An anion-exchange column is used as the ion-exchange column for the labeled reaction product labeled with the labeling reagent (5). A cationic exchange column is used as the ion-exchange column for the labeled reaction product labeled with the labeling reagent (6). The ion-exchange column may normally be composed of a stainless steel column having a length of about 5 to 30 cm and an inner diameter of about 2 to 10 mm. Packing for the anion- or cation-exchange column chromatography may be packed in the empty column or in the column in advance.

The packing for the anion exchange column chromatography may have diethylaminoethyl (DEAE) group or a quaternary ammonium group as a functional group for anionic exchange, and may preferably have DEAE group. The filler for cation exchange chromatography may have sulfopropyl (SP) group or carboxymethyl (CM) group as a functional group for cation exchange, and may preferably have sulfopropyl group. A base material having such ion exchange group includes a polymer gel or silica gel. The polymer gel includes a hydrophilic gel such as polyacrylate series or a hydrophobic gel such as polystyrene series. The base material of the hydrophilic polymer gel suitable for separation of the polyoxyalkylene derivative belonging to a hydrophilic polymer may preferably be used.

The eluent is not limited as far as it is selected among buffer solutions suitable for the separation with an ion-exchange column used. Specifically, the eluent includes formic acid buffer, acetic acid buffer, phosphoric acid buffer, carbonic acid buffer, boric acid buffer, glycine buffer, tris-hydrochloric acid buffer, monoethanolamine-hydrochloric acid buffer etc.. In the case of anion-exchange column, formic acid and acetic acid buffer solutions are preferred and formic acid buffer solution is more preferred. In the case of a cation exchange column, phosphoric acid buffer is preferably used. As the buffer solutions described above, the separation can be effectively performed by selecting the ions and ion-exchange materials so that they have the same electric charge.

Further, either or both of aqueous solution of a salt and/or an organic solvent may optionally mixed with the buffer solution. The aqueous solution of salt includes sodium chloride, potassium chloride, sodium sulfate, potassium sulfate etc.. The organic solvent includes methanol, ethanol, acetonitrile etc., which is easily miscible with water, in a concentration of 0 to 50 volume percent.

The pH of the eluent is not limited in an application range of a column used, and is normally in a range of 2.0 to 12.0. The pH may preferably be 7.0 to 10.0 in the case of anion exchange column, and 4.0 to 8.0 in the case of cation exchange column.

The concentration of salt in the buffer of the eluent is one of important conditions for carrying out the present invention. It is required to set an appropriate concentration of salt in the buffer solution depending on the structure and molecular weight of the polyoxyalkylene derivative. According to an ion-exchange column, the separation is generally performed based on the difference of ionicity of substances. The degree of separation can be further regulated utilizing the concentration of the buffer solution used as the eluent.

In the analytical method according to the present invention, it is studied a concentration of the buffer solution as the eluent required to obtain peak separation performance resulting in excellent quantification and reproducibility. It is thereby proved that an optimum concentration of the buffer correlates with a molecular weight per one functional group of the polyoxyalkylene derivative. That is, the concentration of the buffer solution can be set based on the following formula (p).
y=a/x (p)

In the formula, “y” represents a concentration of the buffer solution (mM), “x” represents a molecular weight per one functional group of the polyoxyalkylene derivative, and “a” represents a numeral in a range of 3000 to 60000. Further, since the ionicity is changed depending on the number of functional groups of the polyoxyalkylene, a preferred range may be set as follows. That is, “a” is preferably 3000 to 30000 in the polyoxyalkylene derivative having one functional group, 15000 to 30000 in the polyoxyalkylene derivative having two functional groups and 30000 to 60000 in the polyoxyalkylene derivative having three or four functional groups. When “a” is smaller than 3000, the concentration of the buffer solution is considerably low so that the contamination and adsorption of the ion-exchange column may occur to result in a reduction of separation.

When the cation exchange column is used for the analysis, “a” is 5000 to 15000 and may preferably be 6000 to 12500. When “a” is lower than 5000, the concentration of the buffer solution is considerably low so that the contamination and adsorption of the ion-exchange column may occur to result in a reduction of separation.

Usually when reversed phase or normal phase column is used for the analysis, it is difficult to perform the separation without applying gradient elution, in which two kinds of eluent compositions are used. According to the analytic method of the present invention, however, it becomes possible to separate polyoxyalkylene derivatives having any molecular weight and structure by isocratic elution using a single eluent, by setting the concentration of the buffer as described above.

According to the analytic method of the present invention, a refractive index detector (RI) is used as an optical detector used in the high performance liquid chromatography. In the case of gradient elution for the analysis, generally, the base line refractive index is changed so that a refractive index detector (RI) is difficult to use. On the contrary, in the case of isocratic elution, a differential refractometer (RI) can be used to calculate the ratio based on the percentage of the corresponding area. As a result, it is not necessary a factor, such as the molecular weight or molar absorbance coefficient of the sample, considerably affecting the analytic value to obtain an accurate value at a high precision. Further when the labeling reagent used has ultraviolet or fluorescent absorbance band, it is preferred to use a ultraviolet detector or a fluorescent detector with the RI. Since ultraviolet and fluorescent detectors have extremely high sensitivity, a trace amount of the terminal activated substance can be detected. Further, when a plurality of peaks are observed on an RI chromatogram, it is possible to identify the peaks of the terminal activated substances based on retention times of the corresponding UV peaks, respectively.

According to the analytic method of the present invention, a single kind of eluent may be preferably used for performing the elution. When a plurality of eluents are used for the elution, however, such plurality of eluents may be used if the change of performance of elution of the used eluents is small. For example, when the eluent is a mixed solvent, it is possible to perform the elution slightly changing the mixing ratio of the mixed solvent. It is further possible to perform the elution with a single eluent and to add a small amount of another solvent to the eluent during the elution.

According to the analytic method of the present invention, the activation ratio of terminal is calculated based on an RI chromatogram obtained by the liquid chromatography. That is, the peaks corresponding to the terminal activated substance and non-activated substance are obtained separately, so that the ratio is calculated based on an area of the peak corresponding to the terminal activated substance.

According to the analytic method of the present invention, when a straight chain polyoxyalkylene derivative having an active group at one terminal contains an activated substance having active groups at both terminals and having a molecular weight larger by two fold than that of the derivative, each of the above activated substances can be separated and quantified. Similarly, when a straight or branched chain polyoxyalkylene derivative has two or more active groups which are structurally symmetrical and equivalent with each other, each of the ratios of the activated substances having different numbers of active groups at the terminals can be separated and quantified.

Further, the labeling reaction easily proceeds in a short time to provide a more stable labeled compound at the terminal active group of the polyoxyalkylene derivative to be analyzed by the inventive analytic method. On the viewpoint, terminal aldehyde, terminal p-nitrophenyl carbamate, terminal maleimide and terminal mercapto groups are preferred and terminal maleimide and terminal aldehyde groups are more preferred.

On the other hand, the ratio is calculated based on the integrated value of each peak corresponding to each hydrogen atom of the terminal active group according to ¹H-NMR method. The sensitivity is lower in sp2 proton than in sp3 proton so that the activation ratio of terminal estimated may be lower than the true value. There is a possibility that the quantification of the measured value of activation ratio of terminal is questioned as maleimide or aldehyde group has sp2 hydrogen atoms. Also on this viewpoint, terminal maleimide and terminal aldehyde groups, among terminal active groups to be analyzed in the method of the present invention, are most preferred for the above additional advantageous effects.

### EXAMPLES

The present invention will be described further in detail, referring to the inventive and comparative examples.

### Example 1

CH₃—(CH₂CH₂O)₂₂₅—CH₂CH₂CHO (6)

20 mg of the above polyoxyalkylene derivative (6) (molecular weight of 10000) was dissolved in 2 mL of 0.1 M buffer solution of acetic acid (pH 4.0). 68 μL of methanol solution (40 mg/mL) of p-nitrobenzoic acid was then added and 128 mL of aqueous solution of sodium cyano borohydride (10 mg/mL) was further added to dissolve the derivative. The mixture was stirred for 2 hours at room temperature to proceed the reaction. The whole of the reaction mixture was added into a gel filtration column (PD-10(Amarsham Bioscience)) equilibrated with eluent used for the subsequent HPLC measurement. Eluent is further added so that a fraction of a high molecular weight eluted first was taken in a vial for HPLC measurement. The HPLC measurement was carried out according to the following conditions.

(Measuring conditions for HPLC measurement)

HPLC system: Alliance 6890 (Waters corporation)

Separation column: ES-502N (Asahipak)

Eluent: 1.5 mM buffer solution of ammonium formate (pH 8.0)

Temperature of column: 30° C.

Flow rate: 1.0 mL/minute

Concentration of sample: 10 mg/mL

Injected amount: 20 μL

Detector: Refractive index detector (RI) (Waters corporation)

UV detector (286 nm) (Waters corporation)

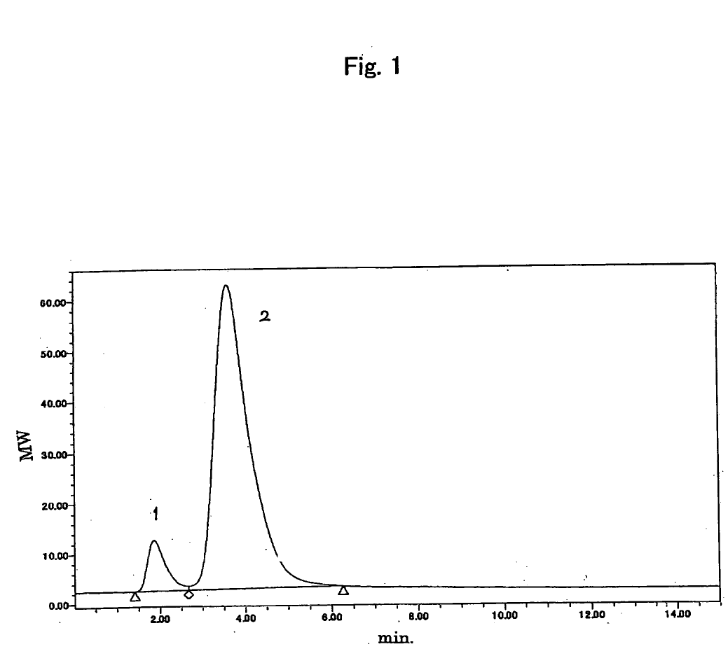

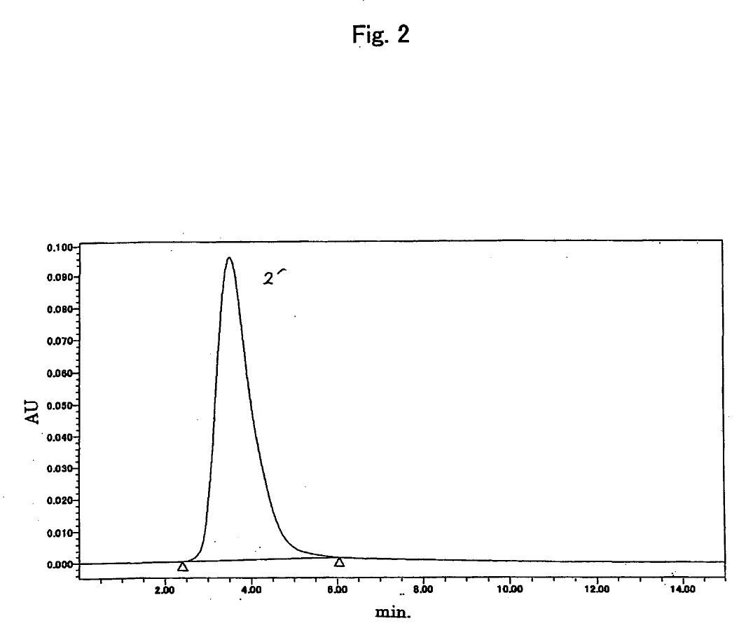

FIGS. 1 and 2 show the RI and UV chromatograms of the measured sample, respectively. Peaks 1 and 2 shown in FIG. 1 correspond with non-activated and terminal-activated substances of the polyoxyalkylene derivative (6), respectively. The identification of the peak 2 as the terminal-activated substance is also confirmed by the fact that a peak 2′ is detected, having the same retention time as the peak 2, in the UV chromatogram shown in FIG. 2. That is, it is possible to specifically detect a component having terminal active group in a sample, by using p-aminobenzoic acid having UV absorbance band as the labeling reagent. The activation ratio of terminal was calculated based on the percentage of the area of the peak 2 and proved to be 91.7 percent.

Further, the above procedure was repeated five times and the reproducibility was measured. The results were shown in table 1.

### Example 2

A sample to be analyzed having a higher molecular weight than in the example 1 used, and the influence on the results and reproducibility of the analytical method were studied.
CH₃O (CH₂CH₂O)₆₈₀—CH₂CH₂CHO (7)

The activation ratio of terminal was measured according to the same procedure as the example 1, for the above polyoxyalkylene derivative (7) having the same structure as the polyoxyalkylene derivative of the example 1 and having a higher molecular weight (molecular weight of 30000). 0.5 mM buffer solution of ammonium formate was used as a eluent for the HPLC measurement. The activation ratio of terminal was proved to be 83.3 percent. The same procedure as described above was repeated and the reproducibility was shown in table 1.

### Comparative Example 1

The activation ratio of terminal of the polyoxyalkylene derivative (6) (molecular weigh of 10000) used in the example 1 was measured by means of ¹H-NMR analysis.

(Conditions for ¹H-NMR measurement)

Equipment: JNM-ECP400

(manufactured by JEOL ITD.) (400 MHz)

Concentration of sample: 20 mg/mL

Heavy solvent: chloroform

Internal standard: TMS

Numbers of integration: 64 times

The thus obtained ¹H-NMR spectrum is used to calculate the activation ratio of terminal based on the ratio of the integrated intensity of a peak assigned to protons of aldehyde group with respect to the theoretical integrated intensity of 1, on the provision that 3 is assigned to the integrated intensity of peaks (3.7 ppm) corresponding to protons of methoxy group. The above procedure was repeated, the reproducibility was measured and the results were shown in table 1.

### Comparative Example 2

The activation ratio of terminal of the polyoxyalkylene deriveative (7) (molecular weight of 30000) used in the example 2 was measured according to the same procedure as the comparative example 1. The procedure was repeated and the results of reproducibility was shown in table 1.

TABLE 1
Results of measurement of activation ratio of terminal by means of the
inventive analytic method and ¹H-NMR method

| | The inventive | | | |
|---|---|---|---|---|
| | analytical method | | ¹H-NMR method | |
| | Example | Example | Comparative | Comparative |
| | 1 | 2 | Example 1 | Example 2 |
| Molecular weight | 10000 | 30000 | 10000 | 30000 |
| of sample | | | | |
| 1 | 91.7 | 83.3 | 86.4 | 77.8 |
| 2 | 91.0 | 83.0 | 90.6 | 66.1 |
| 3 | 92.1 | 83.6 | 87.6 | 76.5 |
| 4 | 92.3 | 83.3 | 89.3 | 66.3 |
| 5 | 91.5 | 83.7 | 88.1 | 85.7 |
| Average value | 91.7 | 83.4 | 88.4 | 74.9 |
| Relative standard | 0.6 | 0.3 | 1.8 | 11.2 |
| deviation (%) | | | | |

The relative standard deviation values (%), indicative of the magnitude of error in the case of repeated measurements, are compared. It is thus proved that the analytical errors are extremely small irrespective of the molecular weight of the analytic sample according to the present invention. In contrast, it is proved that the analytical errors becomes larger as the molecular weight of the sample is higher according to ¹H-NMR method. Further, the activation ratio of terminal was proved to be lower in the ¹H-NMR method than in the inventive analytic method.

According to ¹H-NMR method, as the molecular weight of the sample to be analyzed is larger, the integrated intensity of multiplet peaks (3.8 to 4.3 ppm) assigned to polyoxyethylene chain becomes larger to result in side band noises and the shift of the base line. The integrated intensity of the peak assigned to methoxy group (3.7 ppm) adjacent to the peak assigned to polyoxyethylene chain is thereby influenced to result in an increase of analytical error. Further, the integrated intensity of the peak corresponding to methoxy group may become small due the shift of the base line so that the measured value of the activation ratio of terminal becomes lower than the true value.

As described above, according to the inventive analytical method, the analytical errors are extremely small irrespective of the molecular weight of the sample to be analyzed, unlike ¹H- NMR method, to provide a more suitable method for the quantification of the ratio.

### Example 3

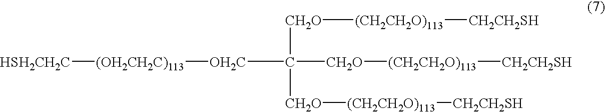

Two samples (7-1, 7-2) of the above polyoxyalkylene derivative (7) (molecular weight of 20000) were measured for the activation ratio of terminals.

20 mg of the above polyoxyalkylene derivative samples (7-1, 7-2) (molecular weight of 20000) were dissolved in 2 mL of maleimide propionic acid aqueous solution (2 mg/mL), respectively. The mixture was stirred for 3 hours at room temperature under shading to provide a sample for measurement. The HPLC measurement was performed according to same procedure as the example 1, except that 1 mM ammonium formate buffer solution (pH 8.0) was used as the eluent.

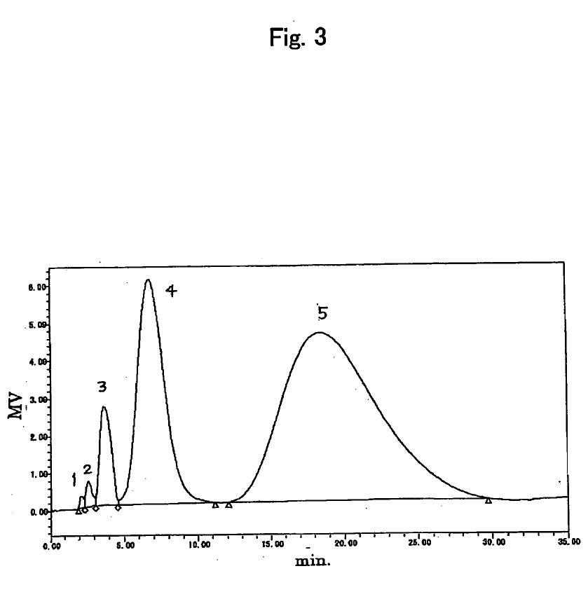

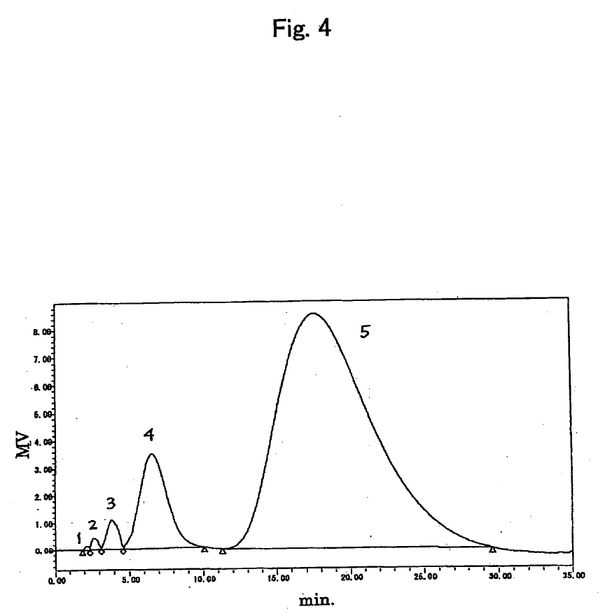

FIGS. 3 and 4 show the RI chromatograms of the samples for measurement (7-1) and (7-2). Peak 1 correspond with a non-activated substance, and peaks 2, 3, 4 and 5 correspond with the polyoxyalkylene derivatives (7) having one, two, three and four active functional groups, respectively. Each of the activation ratios of terminals correspond with the above substances having different numbers of active functional groups based on the percentage of the area of each peak. The activation ratios are added to calculate a total activation ratio of terminal. The results are shown in table 2.

### Comparative Example 3

The activation ratios of terminals of the polyoxyalkylene derivative samples (7-1 and 7-2) used in the example 3 were measured according to the following absorbance spectroscopy method.

The polyoxyalkylene derivative samples (7-1, 7-2) were dissolved in 0.1 M phosphate buffer solution (pH 8.0) so that the concentrations are adjusted at 0.1 mg/mL, respectively (sample solution for test).

L-cysteine was dissolved in 0.1 M phosphate buffer solution (pH 8.0) so that the concentration was accurately adjusted at 0.15 mg/mL (cysteine standard solution). The standard solution was diluted at 100, 50, 25, 16.7 and 12.5 folds, respectively, with 0. 1M phosphate buffer solution (pH 8.0) to provide a series of diluted cysteine standard solutions). Further, 0.1M phosphate buffer solution was used as a blank.

5, 5-dithiobis-2-nitrobenzoic acid (DTNB) was dissolved in 0.1 M phosphate buffer solution (pH 7.0) to adjust the conenctration accurately at 4.6 mg/mL (DTNB solution).

0.25 mL of DTNB solution was added to each of 14.75 mL of the blank, the sample solution for test and a series of the diluted cysteine standard solutions, and held for 10 minutes to perform the derivatization, respectively. Each of the samples after the derivatization was subjected to measurement of absorbance at 410 nm using a spectrophotometer (┌UV-2500PC┘ supplied by SHIMADZU CORPORATION). 0.1 M phosphate buffer solution (pH 8.0) was used as a reference.

Each absorbance corresponding to each concentration of the diluted series of the cysteine standard solutions was plotted to produce a calibration curve, so that the cysteine concentration of the test sample after the derivatization was calculated based on the calibration curve. The calculated cysteine concentration was converted to the activation ratio of terminal based on the molecular weight of the test sample. The results were shown in table 2.

TABLE 2
Results of measurement according to the inventive analytic
method and absorbance spectroscopy method

| | | Contents of substances | | | | | |
|---|---|---|---|---|---|---|---|
| | | having different | | | | | Total |
| | | numbers of functional | | | | Non- | activation |
| | | groups | | | | activated | ratio of |
| | | (%) | | | | substance | terminal |
| | Sample | 4 | 3 | 2 | 1 | (%) | (%) |
| Inventive | 7-1 | 69.5 | 23.8 | 2.8 | 1.6 | 2.3 | 89.1 |
| analytic | 7-2 | 88.1 | 10.4 | 1.3 | 0.0 | 0.2 | 96.6 |
| method | | | | | | | |
| Example 3 | | | | | | | |
| Absorbance | 7-1 | | | | | | 136.0 |
| spectroscopy | 7-2 | | | | | | 122.1 |
| method | | | | | | | |
| Comparative | | | | | | | |
| Example 3 | | | | | | | |

As can be seen from table 2, according to the inventive analytical method, each of the contents of activated substances having the respective numbers of the functional groups among four groups can be quantified, as well as the total activation ratio of terminal. On the contrary, according to the absorbance spectroscopy method, only the total activation ratio can be obtained, and each content of the activated substances having the respective numbers of the functional groups cannot be quantified. It is thus possible to specifically quantify each of the activated substances having the respective numbers of functional groups at terminals of the polyoxyalkylene derivative, according to the inventive analytical method. The inventive method is thus very useful.

Further, the polyoxyalkylene derivative sample (7-1) has a lower activation ratio compared with (7-2). It is thus expected that the synthetic reaction of the polyoxyalkylene derivative is not completed. In this case, raw materials used for the synthesis may be left in the sample.

The reason that the total activation ratio measured by the absorbance spectroscopy method exceeds 100 percent is considered as follows. That is, a mercapto reagent with a low molecular weight, which is a raw material for the synthesis of the polyoxyalkylene derivative, is left in a sample so that the absorbance is considerably increased. As a result, the activation ratio of the sample (7-1) becomes higher in spite of the expectation that the synthetic reaction is not completed in the sample (7-1).

As described above, according to absorbance spectroscopy method, the quantification is adversely affected to prevent accurate measurement of the activation ratio of terminal, when a mercapto substance having a low molecular weight is left in a sample.

### Example 4

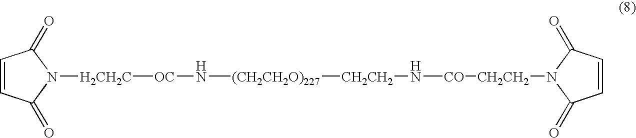

20 mg of the above polyoxyalkylene derivative (8) (molecular weight of 30000) was dissolved in 2 mL of aqueous solution of mercapto propionic acid (1 mg/mL). The solution is stirred under shading for 3 hours at room temperature to provide a sample for measurement. The HPLC measurement was performed according to the same conditions as the example 1, except that 3 mM ammonium formate buffer solution (pH 8.0) was used as the eluent.

Three peaks were found in the RI chromatogram of the sample for measurement. The peaks correspond with a non-activated substance, an activated substance with one terminal activated and an activated substance with both terminals activated in the ascending order of retention time, respectively, and the corresponding ratios were 1.5, 19.5 and 79.0 percent, respectively, based on the percentages of the areas of the peaks.

### Example 5

CH₃O—(CH₂CH₂O)₂₂₅—COC₆H₄NO₂ (9)

The above polyoxyalkylene derivative (9) (molecular weight of 10000) was measured for the distribution of molecular weight according to the GPC measuring conditions.

(GPC Measuring Conditions)

| | |
|---|---|
| Separation column: | PLgel MIXED-D |
| | (Polymer Laboratory) two columns |
| Eluent: | DMF |
| Temperature of column: | 65° C. |
| Flow rate: | 0.7 mL/minute |
| Detector: | RI |
| Concentration of sample: | 1 mg/mL |
| Injected amount: | 100 μL |

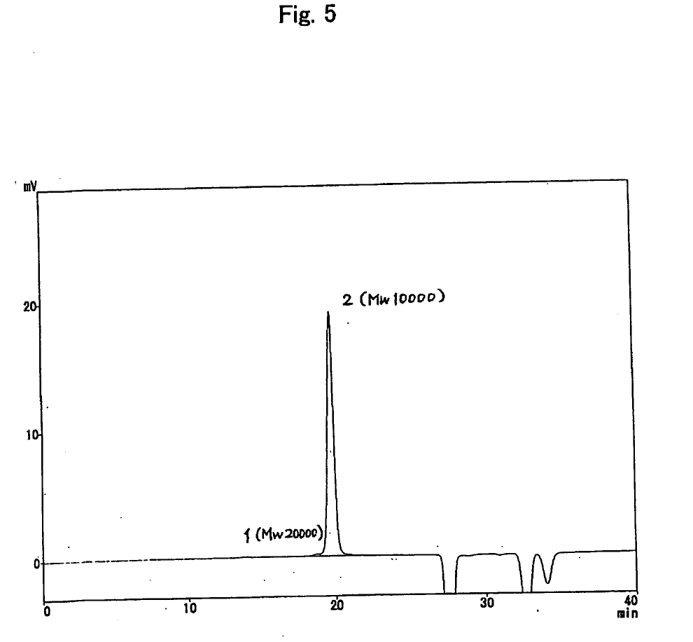

The thus obtained chromatogram was shown in FIG. 5. A peak corresponding with an impurity having a molecular weight of 20000 (two fold) on the left side of a main peak corresponding with a molecular weight of 10000. The impurity was contained in an amount of 0.9 percent. Generally when a straight chain polyoxyalkylene derivative with its terminal occupied with methoxy group is produced, its raw material, methoxy polyoxyethylene, contains a diol (polyoxyethylene having a molecular weight larger by two fold than that of the raw material) as a byproduct in the production of methoxy polyoxyethylene.

The activation ratio of terminal of the polyoxyalkylene derivative (9) was measured according to the following procedure.

10 mg of glycine was added to 50 mg of the above polyoxyalkylene derivative (9) (molecular weight of 10000), and 1 mM of 0.1 M phosphate buffer solution (pH 8.5) was added to dissolve the derivative. The mixture was stirred at room temperature for 20 hours. 2 ml of the reaction mixture diluted five times with a eluent was added into a gel filtration column equilibrated with the eluent used in the HPLC measurement. The eluent was further added thereby to take a fraction of a high molecular weight eluted first in a vial for HPLC measurement. The HPLC measurement was performed according to the same procedure as the example 1, except that the eluent used was 1.5 mM ammonium formate buffer solution (pH 8.0).

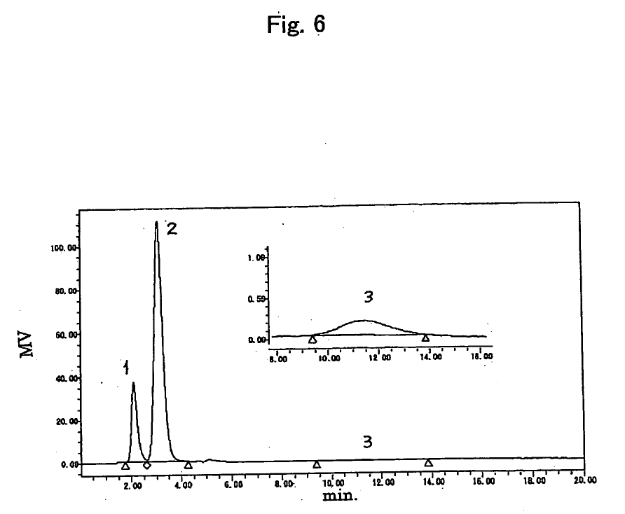

FIG. 6 shows the thus obtained RI chromatogram. The activation ratio of terminal was calculated from the percentage of area of a main peak and proved to be 78.8 percent.

Further, a smaller peak was detected in the back of the main peak. It is speculated that both terminals of polyoxyethylene, having two-fold larger molecular weight, were bonded with active groups based on the retention time. It is thus confirmed that 0.7 percent of the impurity having active groups at both terminals was left among 0.9 percent of the body having two-fold molecular weight confirmed by the above described GPC measurement. As described above, according to the present invention, when a sample contains an impurity of an activated substance having the different molecular weight, it is possible to specifically quantify the impurity.

### Example 6

CH₃O—(CH₂CH₂O)₄₅₄—CH₂CH₂CH₂NHCO-MAL (10)

(MAL: maleimide group)

10 mg of thioethanol amine hydrochloride was added to 40 mg of the polyoxyalkylene derivative (10) (molecular weight of 20000), and dissolved in 2 mL of chloroform. The mixture was stirred at room temperature for 10 hours under shading, and the thioethanol amine hydrochloride not dissolved was filtered out and removed. The reaction mixture was subjected to desalting and dissolved in 8 mL of eluent solution used for the HPLC measurement to provide a sample for measurement. The HPLC measurement was performed according to the following conditions.

(Conditions for Measurement)

| | |
|---|---|
| Separation column: | TSK-gel SP-5PW (Tosoh Corporation) |
| Eluent: | 1.0 mM sodium phosphate buffer solution |
| | (pH 6.5) |
| Temperature of column: | 40° C. |
| Flow rate: | 0.5 mL/minute |
| Detector: | RI |

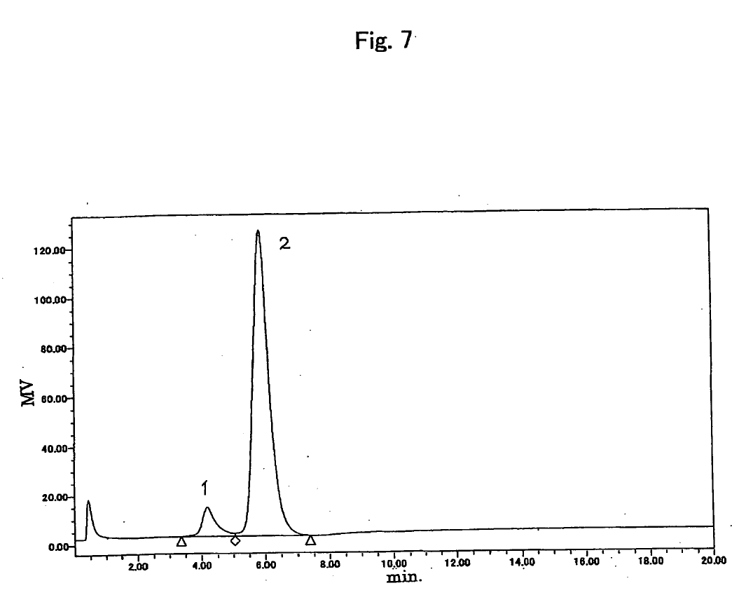

FIG. 7 shows the chromatogram of the sample for measurement. Peak 1 corresponds to the non-activated substance and peak 2 corresponds with the terminal activated substance of the polyoxyalkylene derivative (10). The activation ratio of terminals was proved to be 90.5 percent based on percentage of the area of the peak 2.

1. A method of analyzing an activation ratio of a terminal of a polyoxyalkylene derivative having a terminal active group capable of chemically bonding with a biologically active substance and having a molecular weight of 1000 to 100000, said method comprising the steps of
labeling said terminal active group using a labeling reagent having an ionic functional group;
then analyzing said polyoxyalkylene derivative by means of liquid chromatography using an ion-exchange column and an RI detector outputting a chromatogram; and
obtaining an activation ratio of a terminal based on percentage of an area of a peak in said chromatogram.

2. The method of claim 1, wherein said polyoxyalkylene derivative comprises polyoxyethylene derivative.

3. The method of claim 1, wherein said polyoxyalkylene derivative has a molecular weight of 5000 to 100000.

4. The method of claim 3, wherein said polyoxyalkylene derivative has a molecular weight of 10000 to 100000.

5. The method of claim 1, wherein said polyoxyalkylene derivative comprises a plurality of said terminal active groups.

6. The method of claim 1, wherein said ionic functional group of said labeling reagent comprises carboxyl group and said ion-exchange column comprises an anion-exchange column.

7. The method of claim 1, wherein said ionic functional group of said labeling reagent comprises amino group and said ion-exchange column comprises a cation exchange column.

8. The method of claim 1, wherein said terminal active group of said polyoxyalkylene derivative comprises one or more functional group selected from the group consisting of maleimide and aldehyde groups.

9. The method of claim 1, wherein a buffer solution used as an eluent for said liquid chromatography using said ion exchange column has a concentration (y) satisfying the following formula (p).
y=a/x (p)
(In the formula, “y” represents a concentration of said buffer solution (mM), “x” represents a molecular weight per one functional group of said polyoxyalkylene derivative, and “a” is 3000 to 60000.)

10. The method of claim 9, wherein “a” is 5000 to 15000 in said formula (p).
